(19) United States
(12) Patent Application Publication
(10) Pub. No.: US 2006/0283465 A1
(43) Pub. Date: Dec. 21, 2006

(54) Smart drug delivery system and a method of implementation thereof

(76) Inventors: Janice H. Nickel, Palo Alto, CA (US); Peter G. Hartwell, Palo Alto, CA (US); Sean Zhang, Palo Alto, CA (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 11/155,985

(22) Filed: Jun. 16, 2005

(51) Int. Cl. G06F 19/00; A61K 9/22

(52) U.S. Cl. 128/898; 604/890.1; 705/3; 702/19

(57) ABSTRACT

A smart drug delivery system is disclosed. The system includes a sensor component, an analytical component coupled to the sensor component and a drug delivery component coupled to the analytical component for delivering a drug to a host.

### FIELD OF THE INVENTION

The present invention relates generally to the field of drug delivery and more particularly to a smart drug delivery system and a method of implementation thereof.

### BACKGROUND OF THE INVENTION

Medications can be delivered into the human body by a variety of routes including oral, pulmonary, transdermal, coatings and injections (both intravenous and intramuscular). One of the measures of the effectiveness of a drug is the pharmaco-kinetic (PK) curve. The PK curve of a drug measures the time the dosage of the drug remains in the therapeutic range.

Conventional methods of drug delivery generally results in a rapid absorption of the drug thereby giving spikes in the dosage of the drug which often can exceed the therapeutic range, followed by a rapid fall off of the drug benefit. This causes the effectiveness of the drug to fall below the therapeutic range.

Additionally, as in any therapy, patient compliance is often an issue in that the patient must remember to take the drug at specific times. This can be difficult when the patient has decreased mental faculties due to the ailment or the side effects of the drug therapies.

Some drugs are delivered by time release through an implanted stint. Time released drugs maintain a constant dosage in the body but cannot respond to an increased or decreased need for the drug due to metabolic changes.

Some drugs or drug combinations are only effective when delivered to the specific site where the drugs are needed. For example, ovarian cancer tumors do not respond to chemotherapy administered by IV, but require dosage at the actual site of the tumor.

Some drugs are most effective when given at a specific time period. In this instance, patient compliance plays a large role in the effectiveness of the drug treatment.

Furthermore, in any of the above-described drug delivery scenarios, measurement of the patient's biological data is generally done at the doctor's office. There is no other way to track and record specific biological data (blood sugar level, blood pressure, presence of specific antibodies, etc.) over large periods of time.

Accordingly, what is needed is a method and system that addresses the above delineated problems related to drug delivery. The method and system should be simple, cost effective and capable of being easily adapted to existing technology.

### SUMMARY OF THE INVENTION

A smart drug delivery system is disclosed. The system includes a biological sensory component, an analytical component coupled to the biological sensory component and a drug delivery component coupled to the analytical component for delivering a drug to a host based on the biological sensory component.

Other aspects and advantages of the present invention will become apparent from the following detailed description, taken in conjunction with the accompanying drawings, illustrating by way of example the principles of the invention.

### BRIEF DESCRIPTION OF THE DRAWINGS

FIG. 1 is a flowchart of a method in accordance with an embodiment of the present invention.

FIG. 2 is a block diagram of a system in accordance with an embodiment of the present invention.

FIG. 3 shows a biosensor that could be utilized in conjunction with an embodiment of the present invention.

FIG. 4 shows a drug delivery component that could be utilized in conjunction with an embodiment of the present invention.

FIG. 5 shows an RFID system that could be utilized in conjunction with an embodiment of the present invention.

FIG. 6 shows an energy harvesting system that could be utilized in conjunction with an embodiment of the present invention.

### DETAILED DESCRIPTION

The present invention relates to a smart drug delivery system and a method of implementation thereof. The following description is presented to enable one of ordinary skill in the art to make and use the invention and is provided in the context of a patent application and its requirements. Various modifications to the embodiments and the generic principles and features described herein will be readily apparent to those skilled in the art. Thus, the present invention is not intended to be limited to the embodiment shown but is to be accorded the widest scope consistent with the principles and features described herein.

As shown in the drawings for purposes of illustration, varying embodiments of a smart drug delivery system and method of implementation thereof are disclosed. Accordingly, an actuating drug delivery component is utilized in conjunction with biological sensory components in order to deliver drugs to a host. Consequently, a closed loop drug delivery system is implemented whereby drugs can be delivered to the host based on the dynamic biological needs of the host. As a result, the overall effectiveness of the drug remains in the therapeutic range for a much longer period than that of conventional methodology.

Additionally, the smart drug delivery system includes on-board logic for analyzing the biological information obtained by the biological sensory components. This information can then be utilized to calculate the appropriate drug dosage, archive biological/dosage data, track patient medical data, actuate the drug delivery system, etc.

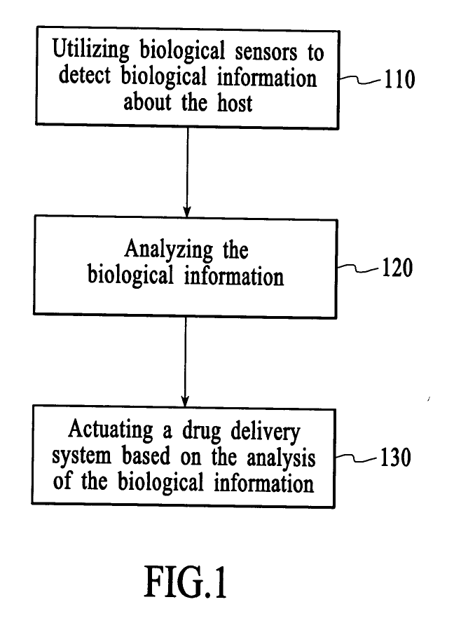

FIG. 1 is a flowchart of a method of delivering drugs to a host. A first act 110 includes utilizing biological sensors to detect biological information about the host. A second act 120 includes analyzing the biological information. A final act 130 includes actuating a drug delivery system based on the analysis of the biological information.

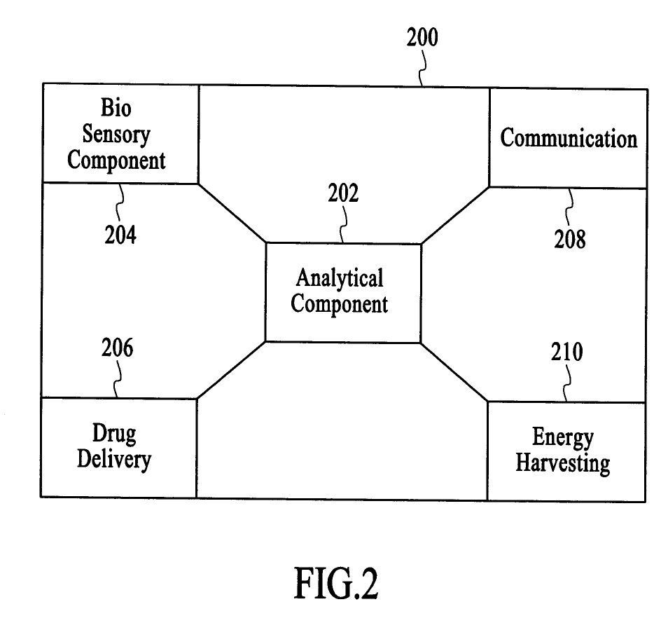

FIG. 2 shows an example of a smart drug delivery system 200 in accordance with an embodiment. The system 200 includes an analytical component 202, a biological sensory component 204, a drug delivery component 206, a communication interface 208 and an energy component 210. In the embodiment of FIG. 2, the analytical component 202 is coupled to the biological sensory component 204, the drug delivery component 206, the communication interface 208 and the energy component 210. However, one of ordinary skill in the art will readily recognize that the components are capable of being coupled together in a variety of ways.

It should be noted that varying embodiments of the smart drug delivery system 200 could be implantable or externally affixed to a biological host. Accordingly, if the smart drug delivery system 200 is implantable, the system 200 could be contained in some type of housing that biological acceptable to the associated host.

Additionally, the smart drug delivery system 200 accordance with an embodiment is designed to be a closed loop drug delivery system. What is meant by closed loop drug delivery is that the system measures biological data, determines a dosage and dispenses the dosage.

Analytical Component 202

In an embodiment, the analytical component 202 includes on-board logic. Logic is the sequence of operations performed by the system. It is the “intelligence” built into the system.

In an embodiment, the on-board logic of the smart drug delivery system 200 works in conjunction with the other components to perform a variety of tasks. First, the logic receives and analyzes biological data from the biological sensory component 204. Based on this data, a predetermined drug delivery algorithm can be utilized to determine a desired drug dosage can be calculated. This algorithm can be based on biological information sensed from the biological sensory component 204 or a variety of other factors. For example, if the biological sensory component 204 senses a particular metabolic change in the host, the on-board logic can calculate a new drug dosage amount based on the metabolic change. Based on the calculated amount of drug, the on-board logic can then send instructions to the drug delivery component 206 to deliver the desired amount of the drug.

In an embodiment, the analytical component 202 also includes a data storage component. Accordingly, the measurement of the metabolic state, and the amount of drug delivered as a function of time can be stored in the data storage component. Furthermore, the data storage component can be utilized to archive dosage data along with other sensed biological data related to the host. This data can later be conveniently transferred to an external hub such as a laptop computer or a personal digital assistant (PDA) containing a medical database via the communication interface 208. Additionally, the analytical component 202 includes some type of timekeeping mechanism (e.g. a clock) to accurately control the timing of each drug dose.

Furthermore, the on-board logic is programmable. Accordingly, if the treating physician needs to alter the predetermined drug delivery algorithm, he/she can do so by re-programming the on-board logic. This is advantageous because it eliminates the need to implant a new device if a new algorithm is necessary.

Although the analytical component is disclosed as being utilized in the above-described fashion, one of ordinary skill in the art will readily recognize that the on-board logic could be utilized to perform a variety of other functions while remaining within the spirit and scope of the presently described concepts. Additionally, the data storage component, as well as the timekeeping mechanism, can be implemented as separate components.

Biological Sensory Component 204

In an embodiment, the biological sensory component 204 includes one or more biological sensors (biosensors). A biosensor is an analytical device that includes at least two components: an immobilized biological component responsible for the selective recognition of the test species and a suitable transducer device responsible for relaying the biological signals for further analysis. Among others, electrochemical biosensors that employ biological recognition systems and electrochemical transduction offer a possibility of quick and real-time analysis, which is particularly suited for the rapid measurement of point-of-care industry.

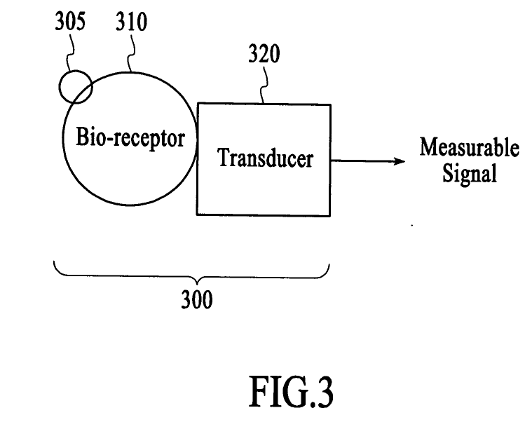

FIG. 3 shows an example of a biosensor 300 that could be used in conjunction with the present embodiment. The biosensor 300 includes bio-receptor 310 and a transducer 320. The bioreceptor 310 could be, for instance, a biomolecule that recognizes a target analyte 305 and the transducer 320 converts the recognition event into a measurable signal. The uniqueness of a biosensor 300 is that the two components are integrated into one single sensor. This combination enables the measurement the target analyte without using reagents. For example, the glucose concentration in a blood sample can be measured directly by a biosensor (which is made specifically for glucose measurement) by simply dipping the sensor in the sample. This is in contrast to the conventional assay in which many steps are used and each step may require a reagent to treat the sample. Accordingly, the simplicity and the speed of measurement are the main advantages of the biosensor 300.

The evolution of these devices comes from the multi-discipline of electronics, material science, electrochemistry, biochemistry, and immunochemistry. The technology of electroanalysis is an interplay between electricity and chemistry that concerns current, potential, and charge from a chemical reaction. There are two principal types of electroanalytical measurements, potentiometric and amperometric. Potentiometric technique is a static technique with no current flow; the established potential across the ion-select membrane is measured. With different types of membrane materials, the recognition of different ions can be reached. Thus, the potentiometric probes have been widely used for directly monitoring ionic species such as calcium, potassium, and fluoride ions.

With the amperometric technique, an electrode potential is used to drive an electron-transfer reaction. The responsive current is measured and related to the presence and/or concentration of the target analyte. In the past, potentiometric devices have been more widely applied in clinical chemistry laboratories. But with increasing amount of research on amperometric systems in diagnostics, the balance has shifted. The amperometric biosensors make possible a practical, fast, and routine measurement of test analysts. The trend of new generations of biosensors focuses on the methodology of minimum demand of operator skills and least sample pretreatment.

Accordingly, an implantable biosensor could be utilized in conjunction with the smart drug delivery system 200 for a variety of potential applications. For example, implantable biosensor(s) could be used to monitor biological systems (e.g. heart rate, breathing rate, etc). Furthermore, biological quantities (e.g. glucose metabolites, white blood cells, etc.) can be monitored/measured. These measurements can then be received and processed by the analytical component 202 and utilized to determine a desirable drug dosage amount and/or a desirable time frame within which to administer the drug.

Although the embodiment is disclosed in the context of being utilized in conjunction with the above-described biosensor, one of ordinary skill in the art will readily recognize that a variety of biosensors could be utilized in conjunction with the embodiment while remaining within the spirit and scope of the presently described concepts.

Drug Delivery Component 206

In an embodiment, the drug delivery component 206 includes a micro-fluidic delivery system that is configured to operate in conjunction with Micro Electro-Mechanical System (MEMS) based valves, reservoirs and/or pumps. Accordingly, the drug delivery component 206 is configured to be operated in response to instructions from the analytical component 202.

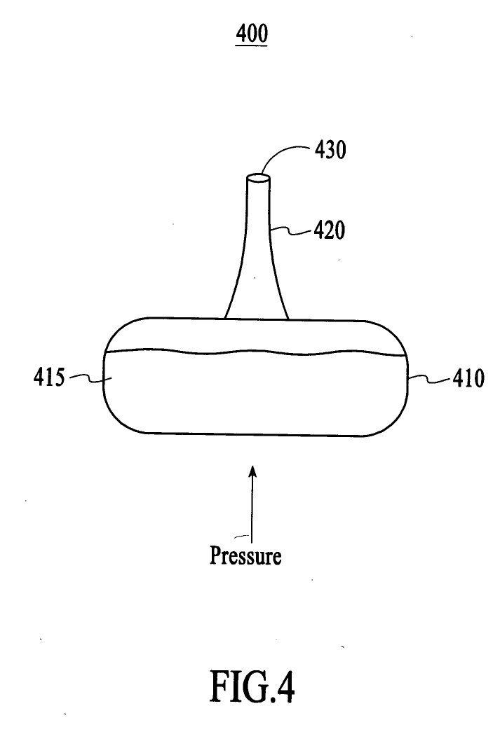

FIG. 4 shows a drug delivery component 400 that could be utilized in conjunction with an embodiment. The component 400 includes a reservoir 410, a micro-needle 420 and a micro-valve 430 whereby the reservoir 410 contains a drug 415 to be delivered to a host. When the drug 415 is ready to be delivered, the analytical component 202 triggers a pressure source to apply a desired pressure to the reservoir 410. This causes the micro-valve 430 to open thereby allowing a desired amount of the drug 415 to flow to the host.

Although the above-described component 400 is implemented with only one drug, it should be readily apparent to one of ordinary skill in the art that multiple reservoirs could be employed to dispense multiple drugs while remaining within the spirit and scope of the disclosed embodiments.

Additionally, the implantable drug delivery component doesn't necessarily need a micro-needle. For example, an alternate embodiment could simply employ an orifice with a hydropholic molecular membrane or other diffusive barrier that can be actuated to deliver the drug.

Communication Interface 208

In an embodiment, the communication interface 208 is a radio-frequency identification communication interface. Radio frequency identification, or RFID, is a generic term for technologies that use radio waves to automatically identify people or objects. There are several methods of identification, but the most common is to store a serial number that identifies a person or object, and perhaps other information, on a microchip that is attached to an antenna (the chip and the antenna together are called an RFID transponder or an RFID tag).

The antenna enables the chip to transmit the identification information to a reader. The reader converts the radio waves reflected back from the RFID tag into digital information that can then be passed on to computers that can make use of the information.

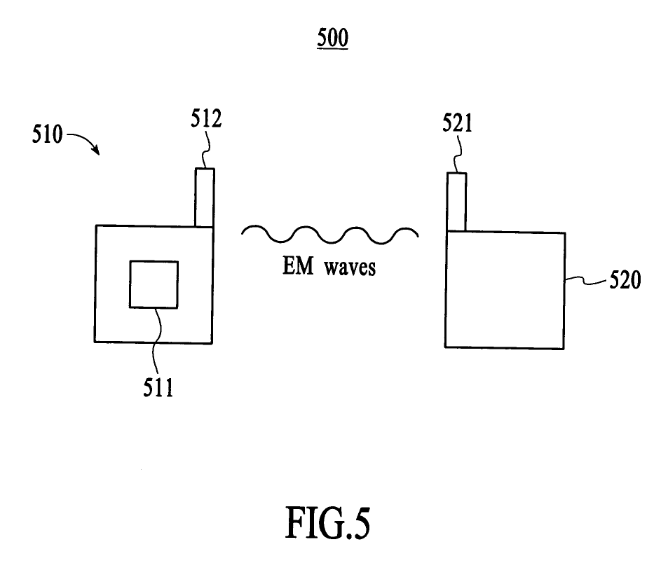

FIG. 5 shows an RFID system 500 that could be utilized in conjunction with an embodiment. The RFID system includes a tag 510, which is made up of a microchip 511 with an antenna 512, and an interrogator or reader 520 with an antenna 521. The reader 520 sends out electromagnetic waves. The tag antenna 512 is tuned to receive these waves. A passive RFID tag draws power from field created by the reader 520 and uses it to power the microchip's circuits. The chip then modulates the waves that the tag 510 sends back to the reader 520 and the reader 520 converts the new waves into digital data.

Accordingly, the RFID system 500 could be implemented to transfer biological data from the analytical component 202 to a personal hub such as a laptop computer or a personal digital assistant (PDA) containing a medical database. Consequently, crucial biological data can be quickly transferred to the medical database with minimal corporation being needed from the patient.

Although the above-described embodiment is disclosed in the context of being utilized with an RFID type communication system, any of a variety of communication system could be implemented. For example, a Bluetooth communication system could be implemented. Bluetooth is an open standard for short-range transmission of digital voice and data between mobile devices and desktop devices. It supports point-to-point and multipoint applications. Unlike Infra-Red, which requires that devices be aimed at each other (line of sight), Bluetooth transmits in the unlicensed 2.4 Ghz band and uses a frequency hopping spread spectrum technique that changes its signal 1600 times per second. If there is interference from other devices the transmission does not stop, but its speed is downgraded.

Additionally, the communication interface 208 is capable of communicating in a two-way fashion. Accordingly, not only can information be extracted from the analytical component 202, information can be transferred to the analytical component 202 as well. For example, if the drug dosage algorithm needs to be altered, the communication interface 202 can be employed to alter the drug dosage algorithm in the desired fashion.

Energy Component 210

In an embodiment, energy to operate the smart drug delivery system can be harvested from converting energy which exists in the host into electrical energy that can be stored for use by the smart drug delivery system. One example of such a system would be an inertial device or a piezoelectric crystal device internally located on a person's muscle or diaphragm. The energy conversion device converts mechanical motion into electrical energy that can be stored in an inert storage device within the system.

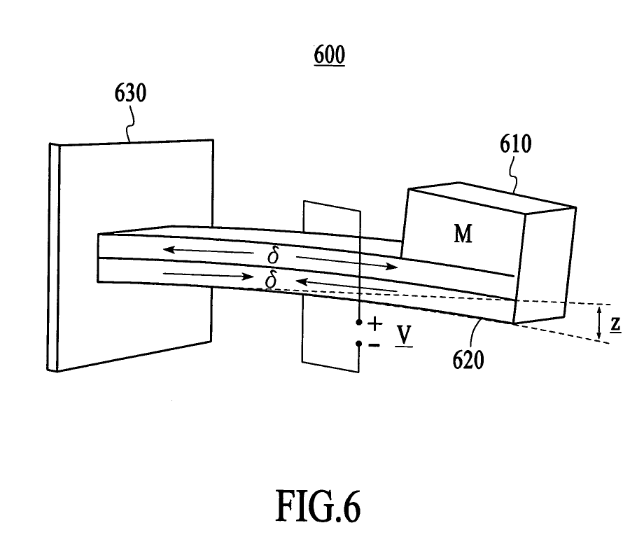

FIG. 6 shows an example of an energy component 600 that could be utilized in conjunction with an embodiment. The component 600 includes a mass 610, a cantilever bending element 620 and electrical conversion circuitry 630. In an embodiment, the cantilever bending element 610 is a piezoelectrical crystal. Accordingly, based on the movement of the cantilever bending element 620 by the mass 610, a voltage is generated that can be converted to electrical energy by the electrical conversion circuitry 630.

Accordingly, a power harvesting and storage system can be designed and embedded in the smart drug delivery system to store and deliver energy from multiple sources of energy harvesting devices. Energy collection is done in parallel whereby energy is stored at a substantially higher rate than energy consumption thereby providing an ample power supply for the smart drug delivery system. Although a battery could be implemented, by employing an energy harvesting component, the battery replacement requirement is removed along with the associated periodic surgeries.

Additionally, if the smart drug delivery system in accordance with an embodiment is externally affixed to the biological host, a solar powered energy component could be utilized in conjunction with the embodiment. Again, this provides a constant energy source and removes the need for batteries.

As shown in the drawings for purposes of illustration, varying embodiments of a smart drug delivery system and method of implementation thereof are disclosed. Accordingly, an actuating drug delivery component is utilized in conjunction with biological sensory components in order to deliver drugs to a host. Consequently, a closed loop drug delivery system is implemented whereby drugs can be delivered to the host based on the dynamic biological needs of the host. As a result, the overall effectiveness of the drug remains in the therapeutic range for a much longer period than that of conventional methodology.

Without further analysis, the foregoing so fully reveals the gist of the present invention that others can, by applying current knowledge, readily adapt it for various applications without omitting features that, from the standpoint of prior art, fairly constitute essential characteristics of the generic or specific aspects of this invention. Therefore, such applications should and are intended to be comprehended within the meaning and range of equivalents of the following claims. Although this invention has been described in terms of certain embodiments, other embodiments that are apparent to those of ordinary skill in the art are also within the scope of this invention, as defined in the claims that follow.

What is claimed:

1. A smart drug delivery system comprising:
a sensor component;
an analytical component coupled to the sensor component; and
a drug delivery component coupled to the analytical component for delivering a drug to a host.

2. The system of claim 1 further comprising:
a communication interface coupled to the analytical component for communicating with an external device.

3. The system of claim 1 further comprising:
an energy harvesting component coupled to the sensor component, the analytical component and the drug delivery component.

4. The system of claim 1 wherein the sensor component further comprises at least one biological sensor.

5. The system of claim 1 wherein the analytical component further comprises on-board logic and storage components for storing and tracking biological data.

6. The system of claim 1 wherein the drug delivery component further comprises a micro fluidic delivery system.

7. The system of claim 1 wherein the system is an implantable system.

8. The system of claim 1 wherein the system is an external system.

9. The system of claim 1 wherein the system is a closed loop system.

10. The system of claim 2 wherein the communication interface further comprises a wireless interface for communicating with the external device.

11. The system of claim 3 wherein the energy harvesting component generates and stores energy based on movement of the host.

12. The system of claim 3 wherein the energy harvesting component comprises a solar-powered energy harvesting component.

13. The system of claim 3 wherein the energy harvesting component comprises a piezoelectric energy harvesting component.

14. The system of claim 4 wherein the at least one biological sensor further comprises a plurality of biological sensors for:
detecting a physiological state of the host;
monitoring at least one biological system of the host; and
monitoring at least one biological quantity within the host.

15. The system of claim 5 wherein the on-board logic is programmable.

16. The system of claim 5 wherein the on-board logic and storage components:
analyze data from the sensor component; and
actuate the delivery component based on the data from the sensor component.

17. The system of claim 6 wherein the micro-fluidic delivery system further comprises MEMS based pumps.

18. The system of claim 6 wherein the micro-fluidic delivery system further comprises MEMS based valves.

19. A method of delivering drugs to a host comprising:
utilizing biological sensors to detect biological information;
analyzing the biological information; and
actuating a drug delivery system based on the analysis of the biological information.

20. The method of claim 19 wherein utilizing biological sensors to detect biological information further comprises:
detecting a physiological state of the host;
monitoring biological systems within the host; and
monitoring at least one biological quantity within the host.

21. The method of claim 19 wherein analyzing the biological information further comprises:
calculating a quantity of drugs to deliver to the host;
determining when to deliver the drugs.

22. The method of claim 19 further comprising:
utilizing a communication interface within the host to transmit the biological information to an external device.

23. The method of claim 19 wherein analyzing the biological information further comprises:
utilizing on-board logic and storage components located within the host to analyze the biological information.

24. The method of claim 19 wherein the drug delivery system further comprises a micro-fluidic delivery system.

25. The method of claim 19 further comprising:
utilizing an energy harvesting component to generate and store energy based on movement of the host.

26. The method of claim 22 wherein the communication interface further comprises a wireless communication interface.
